(19) United States
(12) Patent Application Publication
(10) Pub. No.: US 2021/0338509 A1
(43) Pub. Date: Nov. 4, 2021

(54) PORTABLE SYSTEM FOR PREVENTION OF PATHOGEN TRANSMISSION

(71) Applicant: Joshua Clendenin, (US)

(72) Inventor: Joshua Clendenin, Ackworth, GA (US)

(21) Appl. No.: 16/864,435

(22) Filed: May 1, 2020

(51) Int. Cl. A61G 10/00; A61B 90/50; A61B 46/00; A61B 42/10

(52) U.S. Cl. CPC A61G 10/005; A61B 42/10; A61B 46/40; A61B 90/50

(57) ABSTRACT

The invention discloses a system for protecting healthcare workers from exposure to pathogens while attending to a patient in respiratory distress that requires procedures such as intubation and subsequent placement of a patient on a ventilator. The system comprises a transparent and flexible drape, covering the facial area of the patient with openings situated cranially above the patient, fitted with sleeves/cuffs to which gloves are optionally sealed to the cuffs. The drape is supported above the patient with one or more supporting members, which are secured to the patient's bed. The perimeter of the drape is optionally fitted with a plurality of weights such that when the drape is placed over the supporting member it forms an impervious barrier against pathogens between the patient and the healthcare workers. The system is lightweight and can remain attached to the patient's bed should the patient require movement to a different location.

### FIELD OF THE INVENTION

The invention discloses methods and novel apparatus(es) for protecting medical workers and the surrounding environment from exposure to infectious pathogens while attending to a patient with severe respiratory distress.

### BACKGROUND

The problem of protecting health workers from being infected by the patients they are treating has been made clearly apparent during the COVID-19 pandemic. While personal protective equipment (PPE), namely masks (preferable N95), gowns, face shields, goggles and gloves are effective, the supply of such items may come into short supply. This is especially true because these items are generally one-time use only, so that when the healthcare worker moves from patient to patient, cross contamination is reduced.

Protective measures are particularly important when procedures are undertaken by the healthcare workers that require working in close quarters when treating a patient. An especially hazardous situation is exemplified by the procedure of intubation, that is, inserting a tube into a patient's airway in order to facilitate respiration using a ventilator. In the case of highly contagious diseases, such as the viral infection COVID-19, absent any measures to prevent their spread, the virus particles are easily transmittable over a wide area, requiring decontamination of the workers and the entire patient environment.

An alternative, or at least concurrent method of protection for workers is directed to the use of containment devices to isolate the patient from the worker. To be effective and practical in a pandemic setting, such as COVID-19, such containment devices must be easy to deploy, allow freedom of movement so that procedures can be conducted, and be universally adaptable (“one-size fits all”) to patients of various physical dimensions. Further, because of the sheer volume of patients under pandemic conditions, it would also be advantageous that such a device or system be optionally disposable, or at least, easily removed and quarantined after use.

To date, examples of such devices are typically complex, rigid structures that are highly specialized, cumbersome to setup and use, or lack portability. See for example Kerns et al, U.S. Pat. No. 10,485,620B2; Breegi et al, U.S. Pat. No. 10,251,801B2; Fisher, U.S. Pat. No. 5,316,541; O'Connor et al., U.S. Pat. No. 7,037,254B2; Gnanashanmugam, et al., US 2014/0316455A1; Hyman, U.S. Pat. No. 4,550,713; Forsell, US 2016/0008081; Morledge U.S. Pat. No. 4,223,669; and Mutke, U.S. Pat. No. 4,367,728.

Therefore, there is a further need for systems/devices that are both easily deployable, yet highly effective in preventing exposure of workers as they come in close contact with a patient undergoing a procedure such as intubation.

### SUMMARY OF THE INVENTION

In accordance with the purpose(s) of the invention, as embodied and broadly described herein, the invention, in one aspect, relates to a portable system for protecting healthcare workers from exposure to pathogens while attending to patients in respiratory distress that require procedures such as intubation followed by placement of a patient on a ventilator. The system comprises a transparent and flexible drape, covering the facial area of the patient with openings situated cranially above the patient. The openings are fitted with sleeves/cuffs, into which optional gloves can be inserted, and are sealed to the holes. The drape supported above the patient with a supporting member, such as a lightweight arch-like member, which is optionally secured by a means such as an L or F bracket to the patient's bed. The entire outer perimeter of the drape is fitted on the hem with a plurality of weights such that when the drape is placed over the supporting member it settles down over and beneath the treatment area and once settled, is stationary and forms an effective barrier against pathogens between the patient and the healthcare workers. Alternatively, the drape can be connected to the bed, bed frame or bed rail using hook-and-loop fasteners. The system is lightweight and can remain attached to the patient's bed should the patient require movement to a different location. Embodiments of the system includes both reusable or disposable units.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows an embodiment of the undeployed drape (100) in which weights (110) are configured in the hem (115) at the edge of the drape, and in which two openings (130) in the drape are fitted with sleeves/cuffs (135). The sleeves or cuffs are of a flexible fabric, allowing a medical practitioner to insert gloved hands (right and left) (120) which are sealed to the sleeves/cuffs. When no gloves are used, the sleeves are collapsible and cover the openings, maintaining the barrier between patient and medical worker.

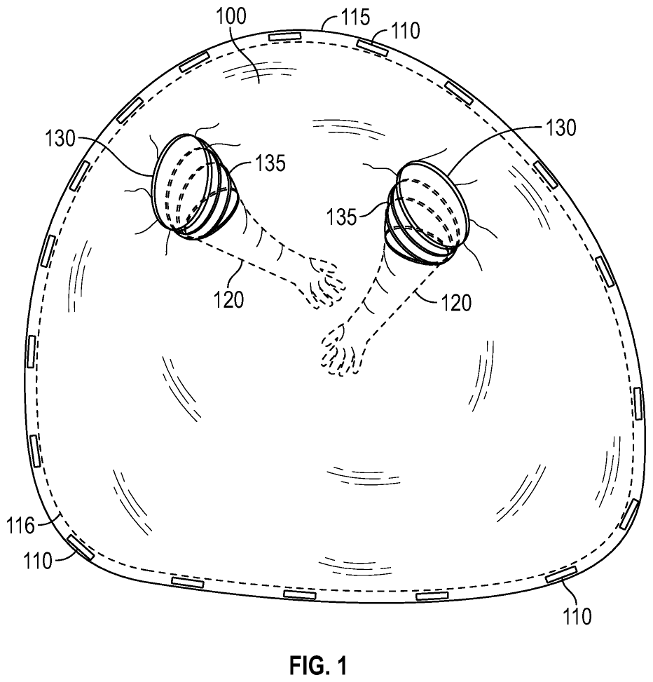

FIG. 2 depicts a perspective side view an embodiment of the deployed drape (200), weighted at the hem (201) with openings on the left (210) and right (220) with corresponding cuffs/sleeves (205), into which are inserted optional gloves (215) and (225) which are sealed to the cuffs/sleeves. The drape is supported over a standard hospital bed by a cylindrically shaped supporting member (230) made of flexible high density foam material, curved above the patient and either physically attached to the bed frame with a bracket or wedged against the bed rail. The drape is configured to cover the entire area near the head/face of the patient, but permits access to the chest area.

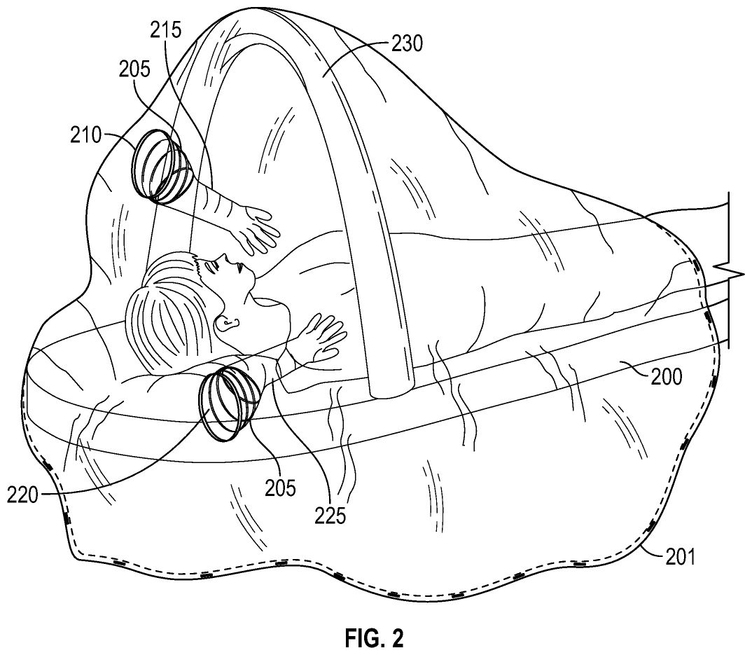

FIG. 3 shows a perspective view of an embodiment of the deployed drape (300), weighted hem (305), glove openings (320) and cuffs with inserted gloves (315), with the supporting member (310) secured to the bed with a bracket (330).

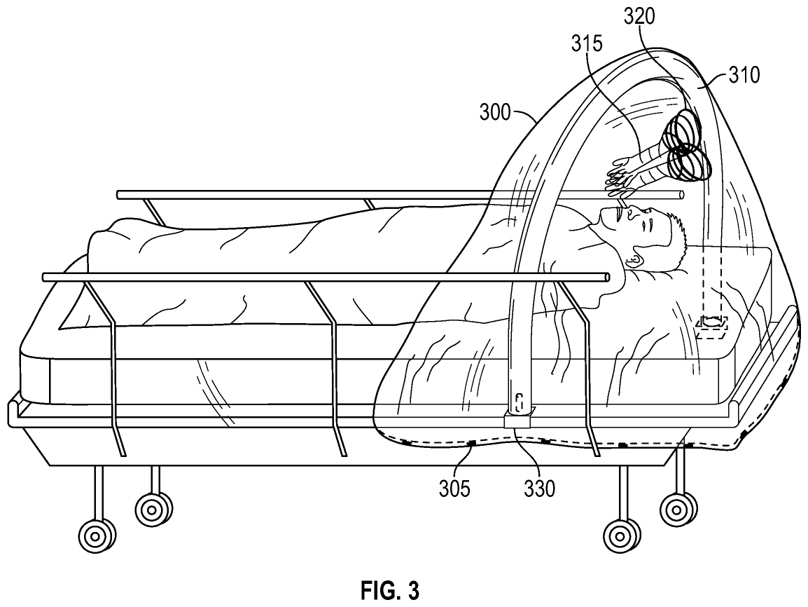

FIG. 4 shows a closeup view of an embodiment in which the supporting member (400) has a hollow core and is configured so that the short end (410) of an L-shaped bracket is inserted into the opening located at the end of the supporting member. The depth to which the bracket is inserted into the opening is adjusted according to the size of the bed and mattress. The longer end of the L-shaped bracket (420) is placed directly underneath the hospital bed mattress. Two such brackets are used in the system, where the longer ends of the brackets are positioned towards each other under the mattress, thus securing the supporting member and drape.

FIG. 5 shows an overhead view of an embodiment of a drape (500), weighted hem (505), glove openings (510), gloves (520), supporting member (535) and bracket (530) attached to the bed frame (540). The drape is deployed over the bed (545) and patient (550), covering the patient's head and neck area.

FIG. 6 shows view from the head end of a patient's (640) bed, showing the supporting member (600) connected to the L-shaped bracket (610) at the attachment point (630), and the longer portion of the bracket (620) anchored under the mattress (650) underside (660).

FIG. 7 is a close up view showing an embodiment in which the supporting member is connected to an F-shaped bracket (700), which is secured to a rail (710) of the bed frame of a hospital bed. The upright portion of the bracket (705) is positioned to accept the end of the supporting member, by inserting in the opening in the end of the member. The two flanges perpendicular to the upright portion of the bracket can be of fixed width as shown, or be adjustable width to accommodate various sizes of bed rails.

FIG. 8 is a closeup view of the hem and perimeter (815) of the drape (800), showing a representative view of the location of weights (820) sewn into the hem (815).

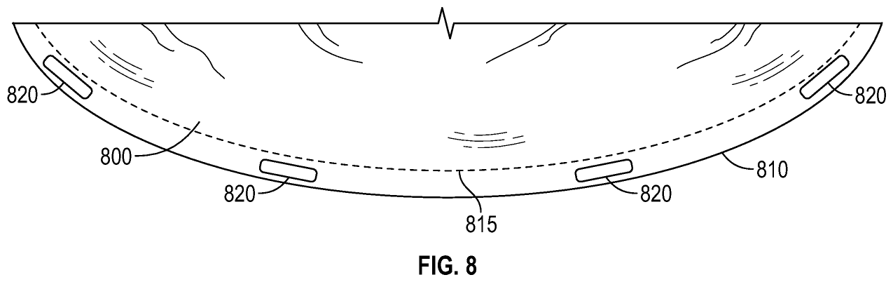

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present invention can be understood more readily by reference to the following detailed description of the invention and the Examples included therein.

Before the compositions, articles, systems, devices, and/or methods are disclosed and described, it is to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

In accordance with the purpose(s) of the invention, as embodied and broadly described herein, the invention, includes a portable and easily adaptable system that comprises a stable yet portable individual protection barrier between a patient who may be discharging harmful pathogens from a respiratory illness such as COVID-19 and the medical workers who are administering treatment and procedures, while also providing safe and ready access to the patient and equipment.

Included in the system is a transparent and flexible drape, covering the facial area of the patient with openings situated cranially above the patient. The material of the drape is selected from any transparent polymeric material and available plastic sheet goods, for example polyethylene, polypropylene, polyvinyl and the like, and is of sufficient thickness, generally between 0.01-4.0 mm, to be resistant to tearing under normal handling by healthcare workers. The material is also selected to be impervious to aerosolized pathogens. In some embodiments, the material is capable of being hemmed, either by sewing or adhesive overlapping, as a means of securing weights around the perimeter of the drape.

The weights can be fitted on the outer perimeter of the drape in one continuous strip of heavier material, e.g. metal wire or strip, or a plurality of weights evenly space around the perimeter. In some embodiments, the configuration of weight about the perimeter of the drape ensures that when the drape is placed over the supporting member (described below), it settles down evenly over the member, with the edge even with or below the bed mattress. In other embodiments where the drape material sufficiently dense, no additional weight is added. Once settled in place, the weighted drape is stationary and forms an effective barrier against pathogens between the patient and the healthcare workers, effectively isolating a potentially contagious patient from those around him or her. The size of the drape is sufficient so that when deployed, covers, at a minimum, the facial area of the patient with the edges of the drape covering the edges of the bed/mattress, so that heath care workers working over or near the patient are protected from any pathogenic particles discharged by the patient. In some embodiments, the drape is large enough to also cover equipment such as a ventilator and associated tubes.

The weights that are sewn in or adhered to the hem of the drape are typically a dense metal, such as iron, steel, lead and the like. The weights add sufficient density to the edge of the drape so that when deployed over the patient, the drape is not susceptible to lifting up or off the patient caused surrounding motion, e.g., while medical procedures are being administered, or by extraneous air movements. In one embodiment, a plurality of the weights are magnetic, placed at multiple points around the drape edge, making the drape edge capable of adhering to the metal of the hospital bed or bedframe, further ensuring a stationary position.

In another embodiment, the edge of the drape is affixed to the bed using a plurality of hook-and-loop fasteners, for example with the hook portion installed on the drape and the loop portion on the bed or a bed frame surface.

It is to be understood that while the drape covers the entirety of the patient's head area, creating an effective shield, it does not form an air-tight seal. Rather, there are slight gaps at the bottom of the drape, well away from the attending healthcare workers, to allow exchange of air to and from the patient, obviating the need to pump in additional air/oxygen into the space covered by the drape. Because of this design, the drape-enclosed space does not develop positive pressure, so there is no undesirable forcing of air and accompanying pathogenic particles from the patient to the outside environment.

The flexible nature of the drape material allows it to be positioned according to the needs of the situation. For example, the drape, can be fabricated to be large enough to provide coverage of varying area sizes. The large, one-size-fits-all, dimensions allow the drape to be adjusted to fit varying dimensions of the patient, as well as any medical devises or equipment, such as a ventilator and associated tubing required during the course of administering treatment to the patient. In addition, drape can be limited so as not to cover the patient's chest area, (i.e., be limited to shoulder level) should access be necessary for other procedures, such as CPR, be necessary.

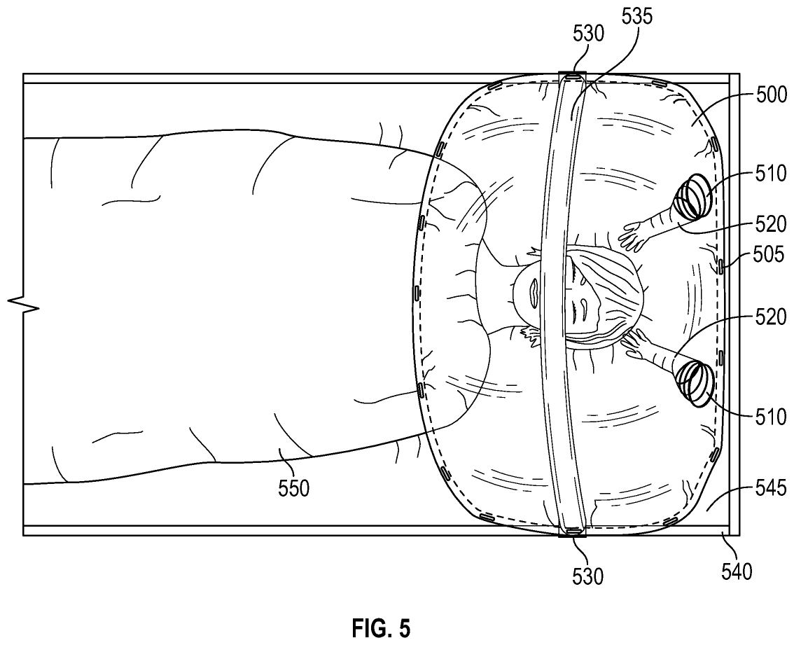

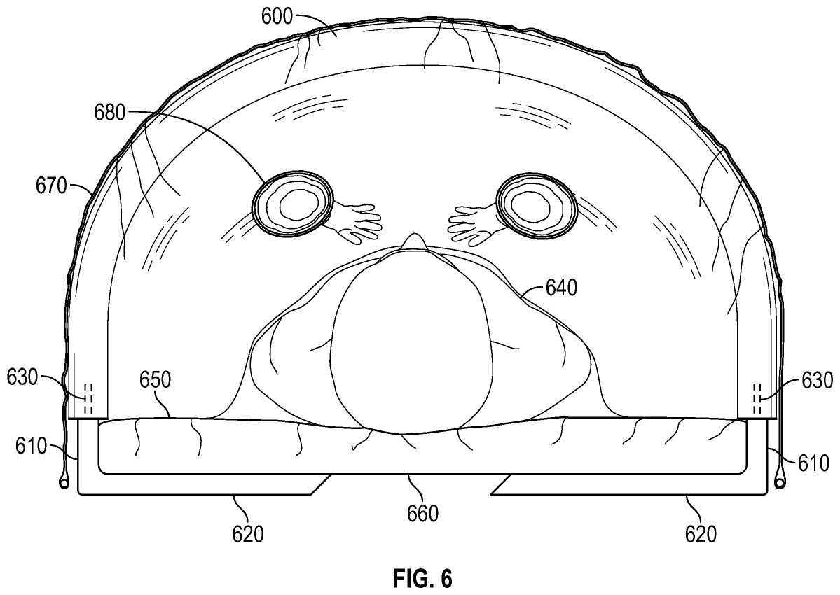

One or more openings are fashioned in the drape to allow access through the drape by the medical worker to the patient, and configured with one or more sleeves and to which are optionally inserted one or more gloves sealed to the sleeves. In representative examples, as depicted by FIGS. 5 and 6, openings are positioned cranially, providing ready access to the mouth and nose area of the patient, and allowing medical personnel to stand in a comfortable position near the patient, so that they may carry out a procedure through the drape wall, such as intubation of the patient and subsequent placement of the patient on a ventilator. In one embodiment the drape has two openings allowing access of gloved hands of one medical worker. In another embodiment, the drape has three holes, allowing access for an additional medical worker.

In a further example, access to the patient is achieved by fitting the openings with sleeves or cuffs into which gloves, or gloved hands can be inserted and sealed to the sleeves/cuffs. The sleeves or cuffs can be constructed using the same material as the drape itself, or can be of any other flexible material fabric, provided that it is resistant to discharged aerosolized pathogens from the patient. It is not necessary for the sleeve or cuff material be transparent. Gloves are fitted into the cuffs or sleeves and attached firmly, by sewing, adhesives, tape, and the like, providing a complete seal with the sleeve and drape. Gloves which are acceptable by medical workers for typical procedures may be used for this purpose, and can be constructed from typical surgical fabrics including rubber, latex, plastic (e.g., polyethylene), nitrile, spun bond olefin fiber, or disposable paper. Selection of the glove is based on the hazard that is expected to be encountered, the dexterity required by the medical staff, and the physical durability required for the material for a specific operation.

In another embodiment, the sleeves, cuffs, and gloves can be manufactured with the same material as the drape and optionally, are manufactured so that drape and gloves are a single continuous surface, topologically shaped to a fit the hands of the medical workers. In this embodiment, drape/glove systems can be manufactured in various sizes, and selected for use by the individual medical worker.

The system includes one or more supporting members, placed across the width of the patient's bed, and over which is placed the drape assembly, providing the space above the patient's facial area allowing the medical worker working space as well as space for any equipment being administered. The members may form an arch, a semicircle, rectangle, or square. The supporting member is optimally placed in a position away from the working area (i.e., away from the patient's face) so as not to hinder the administration of any procedures. In one embodiment, the system consists of one supporting member that is constructed of polyethylene foam, the foam having a density sufficient provide structural stability while retaining sufficient flexibility to achieve the desired shape. Included as examples of acceptable supporting members are cylindrically shaped polyethylene foam materials used either as recreational floatation devices, aka “pool noodles”, or as pipe insulation. In some embodiments, these materials may be hollow, i.e., tubular, possessing a smaller, cylindrically shaped cavity longitudinally through its center.

Other embodiments suitable for use as supporting members include flexible plastic or metal members, including for example, industrial grade plastic rods, flexible fiberglass rods, flexible spring steel, malleable metal rods, high density extruded polystyrene foam, and PVC pipe. In some embodiments, the supporting member may be a single piece, which may optionally be folded for storage. In another embodiment, the supporting member can be sufficiently long and flexible to wrap around the bed frame. In other embodiments, one or more members may be interconnected forming for, example a “cross” shape or “teepee” like framework, which may be assembled and disassembled as needed. In still another embodiment, the supporting member may be an inflatable material, which, when inflated, forms the desired shape of the supporting member (e.g., a cylinder, or a cylinder with a hollow center section).

The supporting member is secured to the bed either by simply wedging it in between the rails of a typical hospital bed, or by one or more fixed accepting means (particularly in the absence of siderails). These include configurations where there is an accepting means mounted on, or proximate to, the bed, mattress, or bed frame.

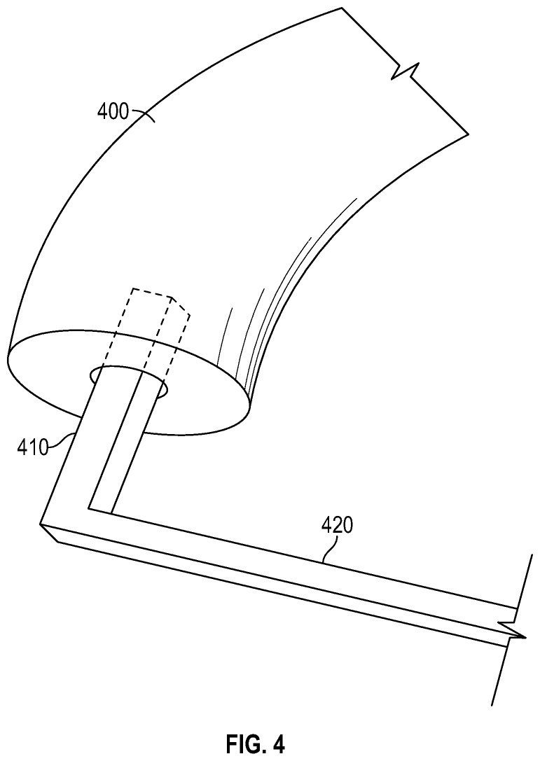

One example of such accepting means is an L-shaped bracket in which one end is attached to one end of the supporting means, e.g., into the hollow portion of the cylindrical foam, and the other end is placed beneath the mattress of the patient's bed, with the weight of the mattress and patient sufficient to keep the bracket in place. Details of this example are illustrated in FIGS. 4 and 6. It is advantageous for the bracket portion under the bed [see FIG. 6, (620)] to be sufficiently long to maintain stability for the supporting member.

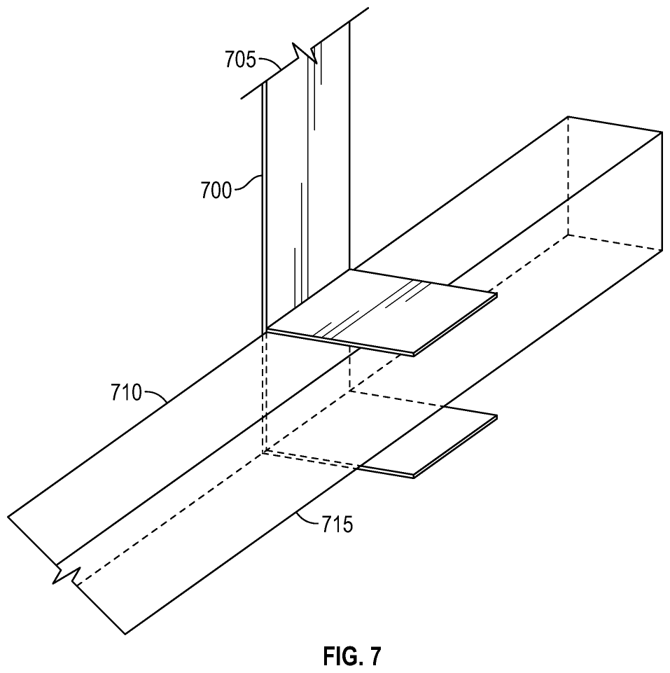

Another example of an acceptable bracket is a so-called F-shaped bracket as appears in FIG. 7. This bracket can be fabricated to fit the particular size of the bed rail being used. Depending on the exact configuration of the bed, this bracket could be attached to the bed rail with the parallel flanges facing inward or outward. In some embodiments, one or both of the flanges of the F-shaped bracket can be adjustable, in order to accommodate various sizes of the bed to which is attached.

Alternatively, brackets or other attachment portions to connected to the supporting member to the bed may be affixed to the bed frame by traditional means, e.g., bolted, screwed, clamped, or welded. Having the bracket already affixed provides the ability to readily and quickly carry out attachment and removal of the drape system.

It can be seen that the system is lightweight and not greatly encumbered by an overabundance of parts and connecting points. As a result it can remain attached to the patient's bed should the patient require movement to a different location in the healthcare unit, but where protection for the healthcare worker is still desired.

In addition, because the system is made up of relatively inexpensive materials and parts, it can either be sanitized, e.g., with aerosolized hydrogen peroxide, or used once and carefully disposed of with little hazard of contamination of the surroundings.

### Definitions

As used in the specification and the appended claims, the singular forms “a,” “an” and “the” include plural referents unless the context clearly dictates otherwise. Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “medical worker(s)”, “health care worker(s)” or “medical staff”, refers to physicians, nurses, medical technologists, or any other workers who are likely to come in contact with patients.

As used herein, the terms “patient(s)” refers to a human person in need of medical attention and being administered to by a medical worker.

As used herein, the term(s) “pathogen” or “pathogenic” refers to any infectious organism capable of causing disease. Such organisms include bacteria, viruses, fungi, and parasites.

As used herein, the term “pathogen transmission” refers to the movement of a pathogen from an infected individual or a contaminated surface to an uninfected individual.

As used herein, the term “enclosure” refers to an volume of space encompassed on all sides with at least one surface. The enclosure may or may not be absolutely sealed from the unenclosed space surrounding it.

As used herein, the term “stationary barrier” refers to a barrier that is not subject to random or accidental movement from where it is intended to be placed.

As used herein, the term “impervious barrier” refers to a barrier comprising a material with the ability to block pathogen transmission, thus preventing the spread of an infection.

As used herein, the terms “hospital bed” or “bed”, refer to any one of a type of standard beds used in health care facilities. Such beds may be configured with or without removable rails.

As used herein the terms “bed frame” and “bed rail” refer to any hardware portions of one may encounter in a typical hospital bed, the size of which may vary depending on the manufacturer and model of the bed.

Specific Aspects

Systems encompassed by this invention include the following aspects:

Aspect 1. A system for prevention of pathogen transmission from a patient to attending medical workers, the system comprising
a transparent drape of polymeric material impervious to pathogenic particles, said material selected from a polyester, polyethylene, polypropylene and polyvinyl polymer;
wherein
   the drape comprises one or more openings, wherein each opening is fitted with a sleeve and into which is optionally inserted a glove that is attached to the sleeves; and
   the perimeter of the drape optionally includes a means of securing the drape to a bed, bed frame or rail, wherein said means is selected from
      a plurality of weights affixed to the edge of the drape wherein one or more weight are magnetic; and hoop-and-loop fasters, attached to the hem of the drape and the bed;
and
one or more supporting members, the supporting members comprising a flexible material selected from polyethylene foam, plastic, and metal;
wherein
   the one or more supporting members are attached to a patient's bed, bed frame or bedrail, span the width of the bed and are configured above the level of the bed in a semicircular, arch or rectangular fashion;
wherein the drape is deployed over the supporting members above the patient's head and shoulder area, the glove/sleeve openings in the drape are positioned above the patient's facial area, and the perimeter of the drape extending to the mattress and sides of the bed, forming an enclosed space above the patient.

Aspect 2. The system of Aspect 1 where the drape comprises two openings.

Aspect 3. The system of Aspect 1 where weights are a metal, selected from steel, magnetic steel and lead.

Aspect 4. The system of Aspect 1 where the drapes is positioned over a single supporting member.

Aspect 5. The system of Aspect 4 where the supporting member is comprised of a cylindrically-shaped polyethylene foam tube.

Aspect 6. The system of Aspect 1 wherein the glove material is selected from rubber, latex, nitrile, polyethylene, spun bond olefin fiber, and disposable paper.

Aspect 7. The system of Aspect 1 wherein the transparent drape material is polyester of about 2 mm in thickness.

Aspect 8. The system of Aspect 5 wherein a supporting member is connected at each end to the bed, bed frame or bed rail with a bracket.

Aspect 9. The system of Aspect 8 wherein the bracket is an L-shaped bracket having a shorter and longer end, the longer end of which is from about 10 to about 18 long.

Aspect 10. The system of Aspect 9 where the long end is secured to the bed by sliding under the mattress.

Aspect 11. The system of Aspect 8 wherein the bracket is an F-shaped bracket.

Aspect 12. The system of Aspect 1 where the volume of the enclosed space accommodates the medical procedure of intubation of the patient and connection to a ventilator.

Aspect 13. The system of Aspect 1 where the pathogenic particles originate from a patient with COVID-19.

Aspect 14. The system of Aspect 1, wherein hook-and-loop fasteners connect the perimeter of the drape to the bed, bed frame or bed rail.

Aspect 15. A system for prevention of pathogen transmission from a patient to attending medical workers, the system comprising
a transparent drape of 2 mm thick polyester; comprising two openings, wherein each opening is fitted with a sleeve and into each sleeve are optionally inserted a glove; and
a plurality of weights are affixed to the perimeter of the drape; and
a supporting member of a hollow polyethylene foam tube of about 52 in long and about 2.5 in wide, with about a 1 in hole, attached to a patient's bed with L-shaped brackets and span the bed in an arch-like fashion;
wherein
the drape is deployed over the supporting member above the patient's head and shoulder area, with the glove/sleeve openings in the drape positioned above the patient's facial area, and the perimeter of the drape extending to the mattress and sides of the bed, forming an enclosed space above the patient.

Aspect 16. A method of preventing the transmission of pathogens from an infected patient to medical workers using the system of claim 1, the method comprising deploying the system over the patient's bed so that the drape of the system encompasses the patient and forms a barrier between the patient and the medical worker while the medical workers are attending to the patient.

Aspect 17. The method of Aspect 16 where the pathogenic particles originate from a patient with COVID-19.

Examples of methods included in the invention are shown below for illustrative purposes and should not be construed as limiting the invention in any way.

### EXAMPLES

### Example 1. Fabrication of a Representative Example

Two holes, approximately 4.5 in in diameter and approximately 12 inches apart, 12 inches from the edge are cut into a 70 in diameter, 2 mm thick 100% transparent polyester sheet. Two sleeves, approximately 12-18 in long and 3-5 in diameter. are fashioned from material from a paper surgical gown and sewn into the holes of polyester to make cuffs.

Approximately 12-15 0.5 to 1 ounce lead weights are hemmed about the entire perimeter of the polyester sheet at regular intervals (spaced about 12 in apart), 12 in).

Into the hollow end (opening is 1 in diameter) of a 52 in long and 2.5 in diameter tubular polyester foam cylinder (aka a “pool noodle”) is placed the short end of a 6 in×12 in L-shaped metal bracket. The cylinder is bent, arch-like fashion, over a standard hospital bed, spanning the bed approximately above the patient's shoulder and neck area, with the long end of each metal bracket placed under the bed mattress, so that the bracket ends are pointing towards each other and the center of the mattress.

The weighted polyester sheet is draped over the foam cylinder support with the sleeve openings placed at the (head) end of the bed, and above the patient's head. The portion of the sheet with the attached sleeves faces towards the patient. The ends of the polyester sheet is allowed to fall down around the patient on the bed, and around the bed such that it rests securely on the mattress and/or on the edge or sides of the bed frame.

### Example 2. Method of Using the Representative System

A patient in need of treatment for a presumed respiratory infection with a contagious pathogen is sedated and placed on a bed. The system described in Example 1 is deployed over the patient and the equipment needed to carry out intubation to the patient and subsequent connection to a ventilator, so that the edges of the drape cover the patient's face and neck, and lay securely on the mattress and against the sides of the bed. The position of the drape is adjusted to ensure that the perimeter of the drape is as far away from the patient's head as possible, while allowing the chest area to remain uncovered. The drape is also situated so that the edge of the drape is secure against the chest of the patient and the sides of the bed, so that the exchange of air from the patient is not directed towards medical workers attending the patient at the head of the patient's bed. A medical worker wearing standard personal protective equipment, inserts gloved hands into the sleeves of the drape and secures them using tape. Using the gloved hands, the medical worker carries out the intubation procedure and connects the patient to ventilator which is then activated. Following the completion of the procedure, if patient/bed is moved to a different location, the system can remain in place as long as needed.

1. A system for prevention of pathogen transmission from a patient to attending medical workers, the system comprising
a transparent drape of polymeric material impervious to pathogenic particles, said material selected from a polyester, polyethylene, polypropylene and polyvinyl polymer;
wherein
the drape comprises one or more openings, wherein each opening is fitted with a sleeve and into which is optionally inserted a glove that is attached to the sleeves; and
the perimeter of the drape optionally includes a means of securing the drape to a bed, bed frame or rail, wherein said means is selected from
a plurality of weights affixed to the edge of the drape wherein one or more weight are magnetic; and hoop-and-loop fasters, attached to the hem of the drape and the bed;
and
one or more supporting members, the supporting members comprising a flexible material selected from polyethylene foam, plastic, and metal;
wherein
the one or more supporting members are attached to a patient's bed, bed frame or bedrail, span the width of the bed, and are configured above the level of the bed in a semicircular, arch or rectangular fashion;
wherein
the drape is deployed over the supporting members above the patient's head and shoulder area, the glove/sleeve openings in the drape are positioned above the patient's facial area, and the perimeter of the drape extending to the mattress and sides of the bed, forming an enclosed space above the patient.

2. The system of claim 1 where the drape comprises two openings.

3. The system of claim 1 where the weights are a metal selected from steel, magnetic steel and lead.

4. The system of claim 1 where the drape is positioned over a single supporting member.

5. The system of claim 4 where the supporting member is comprised of a cylindrically-shaped polyethylene foam tube.

6. The system of claim 1 wherein the glove material is selected from rubber, latex, nitrile, polyethylene, spun bond olefin fiber, and disposable paper.

7. The system of claim 1 wherein the transparent drape material is polyester about 2 mm in thickness.

8. The system of claim 5 wherein the supporting member is connected at each end to the bed, bed frame or bed rail with a bracket.

9. The system of claim 8 wherein the bracket is an L-shaped bracket having a shorter and longer end, the longer end of which is from about 10 in to about 18 in.

10. The system of claim 9 where the long end is secured to the bed by sliding under the mattress.

11. The system of claim 8 wherein the bracket is an F-shaped bracket.

12. The system of claim 1 where the volume of the enclosed space accommodates the medical procedure of intubation of the patient and connection to a ventilator.

13. The system of claim 1 where the pathogenic particles originate from a patient with COVID-19.

14. The system of claim 1, wherein hook-and-loop fasteners connect the perimeter of the drape to the bed, bed frame or bed rail.

15. A system for prevention of pathogen transmission from a patient to attending medical workers, the system comprising
a transparent drape of 2 mm thick polyester; comprising two openings, wherein each opening is fitted with a sleeve and into each sleeve are optionally inserted a glove; and
a plurality of weights are affixed to the perimeter of the drape;
and
a supporting member of a hollow polyethylene foam tube of about 52 in long and about 2.5 in wide, with about a 1 in hole, attached to a patient's bed with L-shaped brackets and span the bed in an arch-like fashion;
wherein the drape is deployed over the supporting member above the patient's head and shoulder area, with the glove/sleeve openings in the drape positioned above the patient's facial area, and the perimeter of the drape extending to the mattress and sides of the bed, forming an enclosed space above the patient.

16. A method of preventing the transmission of pathogenic particles from an infected patient to medical workers using the system of claim 1, the method comprising deploying the system over the patient's bed so that the drape of the system encompasses the patient and forms a barrier between the patient and the medical worker while the medical workers are attending to the patient.

17. The method of claim 16 where the pathogenic particles originate from a patient with COVID-19.
